(19) United States
(12) Patent Application Publication
(10) Pub. No.: US 2009/0130746 A1
(43) Pub. Date: May 21, 2009

(54) MICROCHANNEL SURFACE COATING

(75) Inventor: WEIDONG CAO, ROCKVILLE, MD (US)

Correspondence Address: ROTHWELL, FIGG, ERNST & MANBECK, P.C., 1425 K STREET, N.W., SUITE 800, WASHINGTON, DC, 20005, US

(73) Assignee: CANON U.S. LIFE SCIENCES, INC., ROCKVILLE, MD (US)

(21) Appl. No.: 12/257,064

(22) Filed: Oct. 23, 2008

Related U.S. Application Data
(60) Provisional application No. 60/982,587, filed on Oct. 25, 2007.

(51) Int. Cl. C12M 1/34

(52) U.S. Cl. 435/287.2

(57) ABSTRACT

The present invention relates to a method for improving the efficiency of biochemical reactions in channels of microfluidic devices. More specifically, the present invention relates to the use of chitosan or a chitosan derivative for coating channel surfaces to reduce non-specific adsorption of reagents to microfluidic channels. This reduction of non-specific adsorption improves the efficiency and reproducibility of the reaction, e.g., amplification reactions, such as PCR, and reduces cross-contamination.

### CROSS-REFERENCE TO RELATED APPLICATION

This application claims the benefit of U.S. Provisional Patent Application Ser. No. 60/982,587, filed on Oct. 25, 2007, which is incorporated herein by reference in its entirety.

### BACKGROUND

1. Field of the Invention

The present invention relates to a method for improving the efficiency of biochemical reactions in channels of microfluidic devices. More specifically, the present invention relates to improving the efficiency of biochemical reactions in microfluidic channels by reducing non-specific adsorption of reagents to microfluidic channels thereby improving the efficiency and reproducibility of the reaction, such as, for example, amplification reactions, such as PCR, and reducing cross-contamination.

2. Description of Related Art

The detection of nucleic acids is central to medicine, forensic science, industrial processing, crop and animal breeding, and many other fields. The ability to detect disease conditions (e.g., cancer), infectious organisms (e.g., HIV), genetic lineage, genetic markers, and the like, is ubiquitous technology for disease diagnosis and prognosis, marker assisted selection, correct identification of crime scene features, the ability to propagate industrial organisms and many other techniques. Determination of the integrity of a nucleic acid of interest can be relevant to the pathology of an infection or cancer. One of the most powerful and basic technologies to detect small quantities of nucleic acids is to replicate some or all of a nucleic acid sequence many times, and then analyze the amplification products. PCR is perhaps the most well-known of a number of different amplification techniques.

PCR is a powerful technique for amplifying short sections of DNA. With PCR, one can quickly produce millions of copies of DNA starting from a single template DNA molecule. PCR includes a three phase temperature cycle of denaturation of DNA into single strands, annealing of primers to the denatured strands, and extension of the primers by a thermostable DNA polymerase enzyme. This cycle is repeated so that there are enough copies to be detected and analyzed. In principle, each cycle of PCR could double the number of copies. In practice, the multiplication achieved after each cycle is always less than 2. Furthermore, as PCR cycling continues, the buildup of amplified DNA products eventually ceases as the concentrations of required reactants diminish. For general details concerning PCR, see Sambrook and Russell, Molecular Cloning—A Laboratory Manual (3rd Ed.), Vols. 1-3, Cold Spring Harbor Laboratory, Cold Spring Harbor, N.Y. (2000); Current Protocols in Molecular Biology, F. M. Ausubel et al., eds., Current Protocols, a joint venture between Greene Publishing Associates, Inc. and John Wiley & Sons, Inc., (supplemented through 2005) and PCR Protocols A Guide to Methods and Applications, M. A. Innis et al., eds., Academic Press Inc. San Diego, Calif. (1990).

Real-time PCR refers to a growing set of techniques in which one measures the buildup of amplified DNA products as the reaction progresses, typically once per PCR cycle. Monitoring the accumulation of products over time allows one to determine the efficiency of the reaction, as well as to estimate the initial concentration of DNA template molecules. For general details concerning real-time PCR see Real-Time PCR: An Essential Guide, K. Edwards et al., eds., Horizon Bioscience, Norwich, U.K. (2004).

More recently, a number of high throughput approaches to performing PCR and other amplification reactions have been developed, e.g., involving amplification reactions in microfluidic devices, as well as methods for detecting and analyzing amplified nucleic acids in or on the devices. Microfluidic systems are systems that have at least one channel through which a fluid may flow, which channel has at least one internal cross-sectional dimension, (e.g., depth, width, length, diameter) that typically is less than about 1000 micrometers. Thermal cycling of the sample for amplification is usually accomplished in one of two methods. In the first method, the sample solution is loaded into the device and the temperature is cycled in time, much like a conventional PCR instrument. In the second method, the sample solution is pumped continuously through spatially varying temperature zones. See, for example, Lagally et al. (Analytical Chemistry 73:565-570 (2001)), Kopp et al. (Science 280:1046-1048 (1998)), Park et al. (Analytical Chemistry 75:6029-6033 (2003)), Hahn et al. (WO 2005/075683), Enzelberger et al. (U.S. Pat. No. 6,960,437) and Knapp et al. (U.S. Patent Application Publication No. 2005/0042639).

It is well known that some materials used to prepare microfluidic devices are PCR inhibitory. In addition, it is known that surface adsorption of biological materials, such as proteins and nucleic acids, to the walls of microfluidic channels can cause a variety of problems. For example, in assays relying on flow of material in the channels, adsorption of test or reagent materials to the walls of the channels can cause generally undesirable biasing of assay results. For example, charged biopolymer compounds can be adsorbed onto the walls of the channels, creating artifacts such as peak tailing, loss of separation efficiency, poor analyte recovery, poor retention time reproducibility and a variety of other assay biasing phenomena. The adsorption is due, in part, for example, to electrostatic interactions between, for example, positively charged residues on the biopolymer and negatively charged groups resident on the surface of the separation device. In addition, the adsorption of nucleic acids to the channel walls can lead to contamination that limits the reuse of the microfluidic devices.

Surface passivation techniques have been developed in an attempt to improve the amplification reaction and to reduce cross-contamination. For example, SiO₂ layers have been coated on silicon material surface to block the silicon inhibition on PCR (Krica et al., Anal Biol Chem 377:820-8251 (2003)). Silane reagents such as Sigmacoat® protective coating system are coated on silica surface to decrease the polymerase adsorption on surface (Prakash and Kaler, Microfluid Nanofluid 3:177-1871 (2007)). Some polymers such as PVP have also been tried to dynamically passivate the surface (Kopp et al. Science 280:1047-10481 (1998)). Other coatings, including surface derivatization with poly(ethyleneglycol) and poly(ethyleneimine), functionalization of poly(ethyleneglycol)-like epoxy polymers as surface coatings, functionalization with poly(ethyleneimine) and coating with polyacrylamide, polysiloxanes, glyceroglycidoxypropyl coatings have also been used. See, e.g., Huang et al. (J. Microcol. 4:135-143 (1992)), Bruin et al. (J Chromatogr 471:429-436 (1989)), Towns et al. (J Chromatogr 599:227-237 (1992)), Erim et al. (J Chromatogr 708:356-361 (1995)), Hjerten (J Chromatogr 347:191 (1985)), Jorgenson (Trends Anal Chem 3:51 (1984)) and McCormick (Anal Chem 60: 2322 (1998)). In addition, organic solvent and detergent have been used in an effort to clean the channel to reduce cross-contamination (Liao et al., Biosens Bioelectron 20:1341-13481 (2005)).

Despite the many passivation methods that have been developed, the surface property of microfluidic channels remains a significant challenge for performing biochemical reactions, including PCR, in a microfluidic channel. Although surface treatment by reagents such as silane may decrease surface effect, the passivation layer is not stable during biochemical reactions like PCR, which use high temperatures. Thus, there is a need for a method of reducing non-specific adsorption in microfluidic channels that can withstand the temperatures of PCR without being inhibitory to the reaction.

### SUMMARY OF THE INVENTION

The present invention relates to methods for improving the efficiency of biochemical reactions in channels of microfluidic devices. In one aspect, the present invention relates to the use of chitosan or a chitosan derivative for coating channel surfaces to reduce non-specific adsorption of reagents to microfluidic channels. This reduction of non-specific adsorption improves the efficiency and reproducibility of biochemical reactions, such as, for example, amplification reactions, such as PCR, and reduces cross-contamination.

In accordance with one aspect, the present invention provides methods of reducing non-specific adsorption to a surface of a channel in a microfluidic device. In some embodiments, the method comprises coating the channel surface with a solution comprising chitosan or a chitosan derivative. In other embodiments, the chitosan derivative is chitosan that has been derivatized with a hydrophilic polymer. In further embodiments, the hydrophilic polymer is selected from the group consisting of polyethylene glycol, polyvinyl pyrrolidone, polyvinyl alcohol and poly(methyl methacrylate). In some embodiments, the solution further comprises metal or metal ions. In other embodiments the metal is gold. In some embodiments, the metal or metal ions are in the form of nanoparticles. In other embodiments, the particles have a size of from about 5 nm to about 200 nm. In additional embodiments, the particles have a size of from about 12 nm to about 60 nm.

In other aspects of the present invention, the chitosan or chitosan-derivative is adsorbed to the surface of the microchannel. In some embodiments, the chitosan or the chitosan-derivative has a molecular weight of from about 100,000 daltons to about 5,000,000 daltons. In additional embodiments, the chitosan or the chitosan-derivative has a molecular weight of from about 500,000 daltons to about 5,000,000 daltons. In a further embodiment, the chitosan or chitosan derivative has a molecular weight of about 1,000,000 daltons. In some embodiments, the concentration of the chitosan or chitosan-derivative in the solution is from about 0.01% to about 0.5%. In another embodiment, the concentration of the chitosan or chitosan-derivative in the solution is from about 0.1% to about 0.5%. In other embodiments, the solution further comprises metal or metal ions as described herein. In additional embodiments, the concentration of metal or metal ions in the solution is from about 0.001% to about 0.1%. In further embodiments, the concentration of the metal or metal ions in the solution is from about 0.01% to about 0.05%.

In further aspects of the present invention, the chitosan or chitosan-derivative is covalently bound to the surface of the microchannel. In some embodiments, the chitosan or the chitosan-derivative has a molecular weight of from about 1,000 daltons to about 1,000,000 daltons. In additional embodiments, the chitosan or chitosan-derivative has a molecular weight of from about 1,000 daltons to about 10,000 daltons. In some embodiments, the concentration of the chitosan or chitosan-derivative in the solution is from about 0.1% to about 5%. In additional embodiments, the concentration of the chitosan or chitosan-derivative is from about 1% to about 5%. In other embodiments, the solution further comprises metal or metal ions as described herein. In additional embodiments, the concentration of metal or metal ions in the solution is from about 0.001% to about 0.1%. In further embodiments, the concentration of the metal or metal ions in the solution is from about 0.01% to about 0.05%. In some embodiments, the surface contains a functional group which covalently binds the chitosan or chitosan-derivative. In additional embodiments, the surface is SU-8 polymer which has free epoxy groups to which the chitosan or chitosan derivative covalently binds. In other embodiments, the chitosan or chitosan-derivative is covalently bound to the surface via a linking molecule. In additional embodiments, the linking molecule is 3-glycidoxypropyl-trimethoxy-silane (GTMS) or 3-(trimethoxysilyl)propyl aldehyde (ALDTMS).

In some embodiments, the solution is contacted with the channel for about 1 hour to about 16 hours. In other embodiments, the solution is contacted with the channel at a temperature from about 15° C. to about 70° C. In additional embodiments, the solution is contacted with the channel for about 1 hour to about 8 hours at from about 50° C. to about 70° C. In further embodiments, the solution is contacted with the channel for about 8 hours to about 16 hours at from about 15° C. to about 30° C. In some embodiments, the solution is pulled into the channel for the contacting. In other embodiments, the solution is flushed out of the channel after contacting and the channel is dried.

### BRIEF DESCRIPTION OF THE FIGURES

The accompanying figures, which are incorporated herein and form part of the specification, illustrate various embodiments of the present invention.

FIG. 1 shows a Southern blot of an experiment in which a microchannel was pretreated with water.

FIG. 2 shows a Southern blot of an experiment in which a microchannel was pretreated with chitosan.

FIG. 3 shows a Southern blot of an experiment in which a microchannel was pretreated with chitosan.

### DETAILED DESCRIPTION OF THE PREFERRED EMBODIMENTS

The present invention has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, Oligonucleotide Synthesis: A Practical Approach, 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The present invention relates to a method for improving the efficiency of biochemical reactions in channels of microfluidic devices. In one aspect, the present invention relates to the use of chitosan or a chitosan derivative for coating channel surfaces to reduce non-specific adsorption of reagents to microfluidic channels. This reduction of non-specific adsorption improves the efficiency and reproducibility of the reaction, such as, for example, amplification reactions, such as PCR, and reduces cross-contamination.

The present invention provides methods of reducing non-specific adsorption to a surface of a channel in a microfluidic device, i.e. a microchannel. Microfluidic refers to a system or device having fluidic conduits or chambers that are generally fabricated at the micron to submicron scale, e.g., typically having at least one cross-sectional dimension in the range of from about 0.1 μm to about 500 μm. A microchannel is a channel having at least one microscale dimension. The microfluidic systems are generally fabricated from materials that are compatible with components of the fluids present in the particular experiment of interest. Customarily, such fluids are substantially aqueous in composition, but may comprise other agents or solvents such as alcohols, acetones, ethers, acids, alkanes, or esters. Suitable materials used in the manufacture of microfuidic devices are described, for example, in U.S. Pat. No. 6,326,083 and U.S. Patent Application Publication No. 2007/0246076 A1 and include silica based substrates such as glass and polymeric materials, e.g., plastics, such as polymethylmethacrylate (PMMA), polycarbonate, polytetrafluoroethylene (TEFLON), polyvinylchloride (PVC), polydimethylsiloxane (PDMS), polysulfone, polystyrene, polymethylpentene, polypropylene, polyethylene, polyvinylidine fluoride, ABS (acrylonitrile-butadiene-styrene copolymer), epoxy type polymers (such as SU-8, a negative, epoxy-type, near-UV photoresist), and the like. Such polymeric substrates are readily manufactured using available microfabrication techniques or from microfabricated masters, using well known molding techniques.

In one embodiment, the method comprises coating the channel surface with a solution comprising chitosan or a chitosan derivative. Chitosan, as used herein, is at least partially deacetylated chitin. In some embodiments, the degree of deacetylation ranges from between 30% and 99.5%. In other embodiments, the degree of deacetylation is at least 50%. In additional embodiments, the degree of deacetylation is at least 75%. In further embodiments, the degree of deacetylation is at least 85%.

Chitosan is a polysaccharide poly-[1,4]-β-D-glucosamine that comes commercially in a variety of forms including, but not limited to, mixtures of different weight molecules, which range from about 1,000 daltons to more than 5,000,000 daltons. Different molecular weights of chitosan are useful in different embodiments of the present invention, as described in further detail herein. The chitosan derivative is chitosan that has been derivatized with a hydrophilic polymer to improve the hydrophilicity characteristics of the chitosan. The chitosan-derivatives are prepared as is known in the art. Examples of suitable hydrophilic polymers that can be used to make the chitosan derivative include polyethylene glycol, polyvinyl pyrrolidone, polyvinyl alcohol and poly(methyl methacrylate). Commercial preparations of chitosan or chitosan-derivatives can be used in the practice of the present invention. In some embodiments, the solution further comprises metal or metal ions. In other embodiments the metal is gold, silver, copper, zinc or platinum. In some embodiments, the metal or metal ions are in the form of nanoparticles. In other embodiments, the nanoparticles have a size of from about 5 nm to about 200 nm. In additional embodiments, the nanoparticles have a size of from about 12 nm to about 60 nm.

In one embodiment, the chitosan or chitosan-derivative is adsorbed to the surface of the microchannels. For this embodiment, the chitosan or the chitosan-derivative has a molecular weight of from about 100,000 daltons to about 5,000,000 daltons. In additional embodiments, the chitosan or the chitosan-derivative has a molecular weight of from about 500,000 daltons to about 5,000,000 daltons. In a further embodiment, the chitosan or chitosan derivative has a molecular weight of about 1,000,000 daltons. In some embodiments, the concentration of the chitosan or chitosan-derivative in the solution is from about 0.01% to about 0.5%. In another embodiment, the concentration of the chitosan or chitosan-derivative in the solution is from about 0.1% to about 0.5%. If too high a concentration of chitosan or chitosan-derivative is used, the chitosan or chitosan-derivative could precipitate out of solution so that a uniform coating on the microchannels is not achieved. The solvents used for preparing the solution of chitosan or chitosan-derivative are well known in the art for this molecular weight range. In one embodiment, the solvent is acetic acid. In other embodiments, the solution further comprises metal or metal ions as described herein. In additional embodiments, the concentration of metal or metal ions in the solution is from about 0.001% to about 0.1% and may be in the form of nanoparticles as described herein. In further embodiments, the concentration of the metal or metal ions in the solution is from about 0.01% to about 0.05%.

In a second embodiment, the chitosan or chitosan-derivative is covalently bound to the surface of the microchannel. For this embodiment, the chitosan or the chitosan-derivative has a molecular weight of from about 1,000 daltons to about 1,000,000 daltons. In additional embodiments, the chitosan or chitosan-derivative has a molecular weight of from about 1,000 daltons to about 10,000 daltons. In some embodiments the concentration of the chitosan or chitosan-derivative in the solution is from about 0.1% to about 5%. In additional embodiments, the concentration of the chitosan or chitosan-derivative is from about 1% to about 5%. If too high a concentration of chitosan or chitosan-derivative is used, the chitosan or chitosan-derivative could precipitate out of solution so that a uniform coating of the microchannels is not achieved. The solvents used for preparing the solution of chitosan or chitosan-derivative are well known in the art for this molecular weight. In one embodiment, the solvent is water. In other embodiments, the solution further comprises metal or metal ions as described herein. In additional embodiments, the concentration of metal or metal ions in the solution is from about 0.001% to about 0.1% and may be in the form of nanoparticles as described herein. In further embodiments, the concentration of the metal or metal ions in the solution is from about 0.01% to about 0.05%.

In some embodiments, the surface of the microchannel contains a functional group which covalently binds the chitosan or chitosan-derivative. For example, the microchannels can be made from SU-8 polymer and, as a result of the fabrication, the SU-8 polymer at the surface of the microchannels contains free epoxy groups. In an alternative example, the microchannels can be made from glass and SU-8 polymer can be layered over the glass to provide free epoxy groups at the surface of the microchannels. The chitosan or chitosan derivative reacts with the free epoxy groups to be covalently linked to the surface. In other embodiments, the chitosan or chitosan-derivative is covalently bound to the surface via a linking molecule. In some embodiments, the linking molecule is 3-glycidoxypropyl-trimethoxy-silane (GTMS). For example, the microchannels can be fabricated in glass. The glass surface can be treated with silanes having dual functional groups, such as GTMS, as is well known in the art. The chitosan or chitosan-derivative then reacts, for example, through its reactive amino groups, with those functional groups of the linking molecule not covalently bound to the glass surface. In other embodiments, the linking molecule is 3-(trimethoxysilyl)propyl aldehyde (ALDTMS). The glass surface can be treated with ALDTMS to functionalize the surface with aldehyde groups. The chitosan or chitosan derivative is bound to the modified glass surface through the aldehyde groups.

In one embodiment, the solution of chitosan or chitosan-derivative is contacted with the channels of the microfluidic device for about 1 hour to about 16 hours. The dwell time of the solution in the microchannels for properly coating the channels is pH and temperature dependent. A dwell time of 1-2 hours is preferred to provide a uniform coating on the microchannels. Longer dwell times are useful to ensure that a uniform coating is obtained, especially in those embodiments in which the chitosan or chitosan-derivative are adsorbed to the surface. In some embodiments, the pH of the solution is from about 4.0 to about 10.0, preferably from about 6.0 to about 8.0, more preferably about 7.0. In other embodiments, the solution is contacted with the channel at a temperature from about 15° C. to about 70° C. Generally, the lower the temperature, the longer the dwell time required to obtain a uniform coating on the microchannel surfaces. In additional embodiments, the solution is contacted with the channel for about 1 hour to about 8 hours at from about 50° C. to about 70° C. In further embodiments, the solution is contacted with the channel for about 8 hours to about 16 hours at from about 15° C. to about 30° C. In some embodiments, the solution is pulled into the channel for the contacting. In other embodiments, the solution is flushed out of the channel after contacting and the channel is dried prior to use.

Once the channels of the microfluidic device have been coated with the chitosan or chitosan-derivative (optionally containing metal or metal ions), by adsorption or covalently, the device is ready to be used for those reactions for which such devices are used as is well known in the art. One such use of microfluidic devices having microchannels coated in accordance with the present invention is to run PCR reactions and/or to perform thermal melts in the microchannels. It has been found that the coatings are stable to the high temperatures and the temperature cycling used for PCR reactions and to the upper temperatures used for thermal melts. The chitosan or chitosan-derivative coatings of the present invention do not peal off of the microchannel surfaces during the amplification reaction.

An experiment was conducted in which the microchannels of a microfluidic chip were pretreated with water by flowing water through the microchannels. The microfluidic chip was a pattern 2, type 2 chip in which the microchannels were prepared with SU-8 having a depth of 7 μm. The microfluidic chip is a SU-8-bond glass chip. The channel size is 20 mm long, 180 μm wide and 20 μm deep. Microfluidic chips made with SU-8 have free epoxy groups at the surfaces of the microchannels.

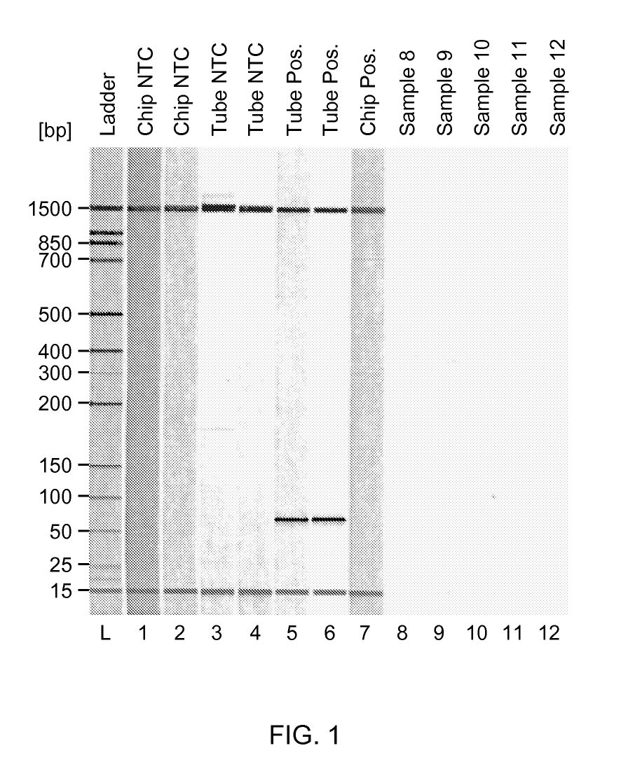

PCR amplification reactions were performed in microchannels (and eppendorf tubes for control) as follows. A PCR amplification of human genomic DNA was performed using primers for a sickle assay. The primers that were used have the sequences: forward: 5′ CAACTTCATCCACGTTCACC 3′ (SEQ ID NO:1) and reverse: 5′ ACACAACTGTGTTCACTAGC 3′ (SEQ ID NO:2). The PCR reaction contained 1×PCR buffer, 0.2 mM dNTPs, 2 mM MgCl₂, 0.5 μM of forward and reverse primers, 1× LCGreen dye, 0.1 unit/μL Taq polymerase, 1 M Betaine, 0.04% Tween-20, 2% DMSO, 5 ng/μL sample DNA in a total volume of 20 μL or 200 μL. The reaction mixture was heated to 95° C. for two minutes and then cycled 40 times at 95° C. for 20 seconds, 60° C. for 20 seconds and 72° C. for 20 seconds. The reaction mixture was maintained at 72° C. for 2 minutes after the last cycle and then cooled to 4° C. before analysis. The results of this experiment are shown in FIG. 1.

FIG. 1 shows a Southern blot of an experiment in which the microchannel was pretreated with water. The lane designations are as follows: Ladder—molecular weight markers; Chip NTC—microfluidic chip with no DNA template; Tube NTC—eppendorf tube with no DNA template; Tube Pos.—eppendorf tube with DNA template; and Chip Pos.—microfluidic chip with DNA template. As shown in FIG. 1, amplification of the template (band between 50 and 100 on the gels) occurred in the eppendorf tubes (the control) that contained the template. No amplification occurred in the microchannels of the chip which were pretreated with water. These results show that the surface of the microchannels adsorbs the components of the PCR reaction mixture preventing the amplification of the template nucleic acid in the microchannels.

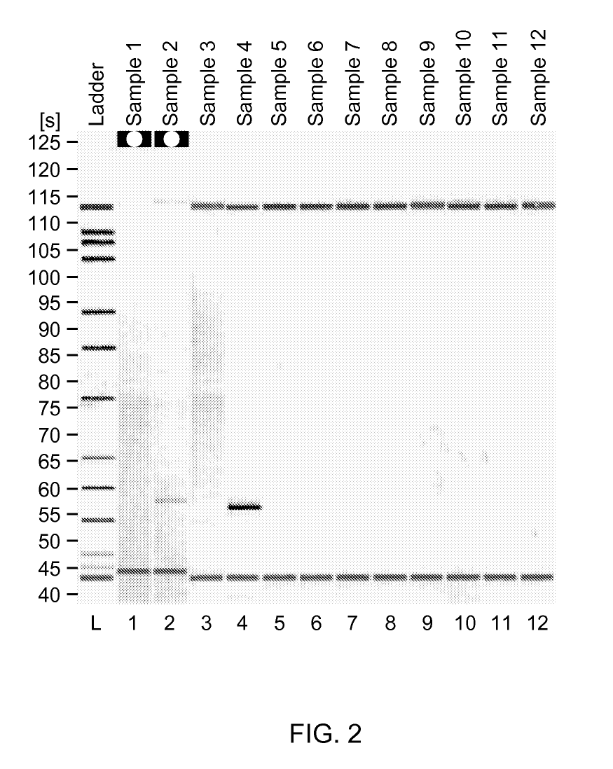

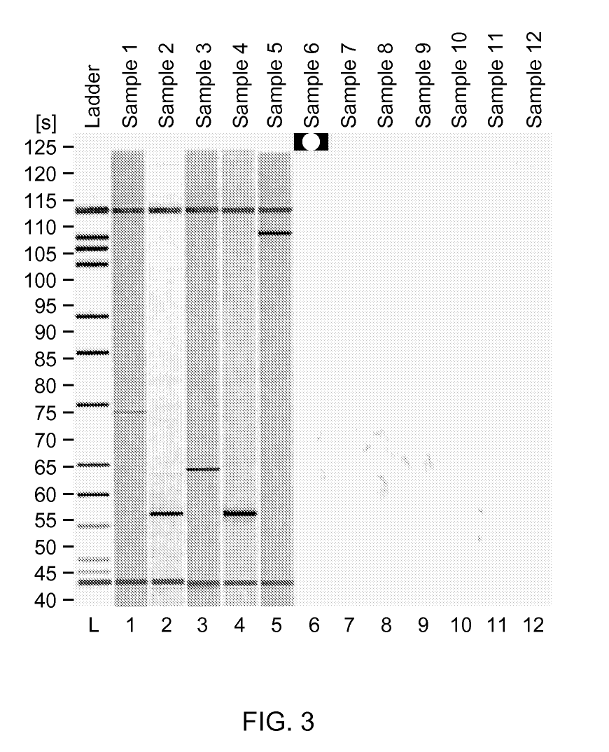

An experiment was conducted in which the microchannels of a microfluidic chip were pretreated with a chitosan solution. The microfluidic chips were pattern 2, type 2 chips in which the microchannels were prepared with SU-8 having a depth of 7 μm. Chips made with SU-8 have free epoxy groups at the surfaces of the microchannels. For the pretreatment of the chips, a 5% chitosan solution was prepared by dissolving chitosan oligosaccharide lactate (Mₙ<5000; Sigma). The microchannels of the chip were filled with the 5% chitosan solution. The reservoir containing the chitosan solution was covered with mineral oil. The chips were placed in an oven at 75° C. overnight (more than 12 hours). The chitosan solution was removed and the microchannels were cleaned with isopropyl alcohol, water and finally 20 mM Tris buffer (pH 9). PCR amplification reactions were performed in the chitosan-treated microchannels and in control eppendorf tubes as described above. The results of this experiment are shown in FIGS. 2 and 3.

FIG. 2 shows a Southern blot of an experiment in which a microchannel was pretreated with chitosan. The lane designations are as follows: Ladder—bp size markers; Sample 1—chip with no DNA template; Sample 2—Chip with DNA template; Sample 3—eppendorf tube with no DNA template; Sample 4—eppendorf tube with DNA template. FIG. 3 shows a Southern blot of an experiment in which the microchannel was pretreated with chitosan. The lane designations are as in FIG. 2.

As shown in FIGS. 2 and 3, amplification of the template (band between 55 s and 60 s on the gels) occurred in the eppendorf tubes pretreated with chitosan that contained the template and in the microchannels pretreated with chitosan that contained the template. These results show that pretreatment with chitosan prevents the adsorption of the components of the PCR reaction mixture, e.g., polymerase and nucleic acid template, to the surface of the microchannels.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of improving the efficiency of a biochemical reaction in a channel in a microfluidic device, said method comprising coating the channel surface with a solution comprising chitosan or a chitosan derivative to reduce non-specific adsorption to a surface of the channel.

2. The method of claim 1, wherein the biochemical reaction is an amplification reaction.

3. The method of claim 2, wherein the amplification reaction is a polymerase chain reaction.

4. The method of claim 1, wherein the chitosan derivative is chitosan that has been derivatized with a hydrophilic polymer.

5. The method of claim 4, wherein the hydrophilic polymer is selected from the group consisting of polyethylene glycol, polyvinyl pyrrolidone, polyvinyl alcohol and poly(methyl methacrylate).

6. The method of claim 1, wherein the solution further comprises metal or metal ions.

7. The method of claim 6, wherein the metal is selected from the group consisting of gold, silver, copper, zinc and platinum.

8. The method of claim 6, wherein the metal or metal ions are in the form of nanoparticles.

9. The method of claim 8, wherein the particles have a size of from about 5 nm to about 200 nm.

10. The method of claim 9, wherein the particles have a size of from about 12 nm to about 60 nm.

11. The method of claim 6, wherein the metal is gold.

12. The method of claim 6, wherein the concentration of metal or metal ions is from about 0.001% to about 0.1%.

13. The method of claim 12, wherein the concentration of metal or metal ions is from about 0.01 to about 0.05%.

14. The method of claim 1, wherein the chitosan or chitosan-derivative is adsorbed to the surface.

15. The method of claim 14, wherein the chitosan or the chitosan-derivative has a molecular weight of from about 100,000 daltons to about 5,000,000 daltons.

16. The method of claim 15, wherein the chitosan or the chitosan-derivative has a molecular weight of from about 500,000 daltons to about 5,000,000 daltons.

17. The method of claim 16, wherein the chitosan or the chitosan-derivative has a molecular weight of about 1,000,000 daltons.

18. The method of claim 15, wherein the concentration of the chitosan or chitosan-derivative in the solution is from about 0.01% to about 0.5%.

19. The method of claim 18, wherein the concentration of the chitosan or chitosan-derivative in the solution is from about 0.1% to about 0.5%.

20. The method of claim 1, wherein the chitosan or chitosan-derivative is covalently bound to the channel surface.

21. The method of claim 20, wherein the chitosan or the chitosan-derivative has a molecular weight of from about 1,000 daltons to about 1,000,000 daltons.

22. The method of claim 21, wherein the chitosan or the chitosan-derivative has a molecular weight of from about 1,000 daltons to about 10,000 daltons.

23. The method of claim 20, wherein the concentration of the chitosan or chitosan-derivative in the solution is from about 0.1% to about 5%.

24. The method of claim 23, wherein the concentration of the chitosan or the chitosan-derivative in the solution is from about 1% to about 5%.

25. The method of claim 20, wherein the surface contains a functional group which covalently binds the chitosan or chitosan-derivative.

26. The method of claim 25, wherein the functional group is an epoxy group on the surface.

27. The method of claim 20, wherein the chitosan or chitosan-derivative is covalently bound to the channel surface via a linking molecule.

28. The method of claim 27, wherein the linking molecule is 3-glycidoxypropyltri-methoxysilane (GTMS) or 3-(trimothoxysilyl)propyl aldehyde (ALDTMS).

29. The method of claim 1, wherein the solution is contacted with the channel for about 1 hour to about 16 hours.

30. The method of claim 29, wherein the solution is contacted with the channel at a temperature from about 15° C. to about 70° C.

31. The method of claim 30, wherein the solution is contacted with the channel for about 1 hour to about 8 hours at from about 50° C. to about 70° C.

32. The method of claim 30, wherein the solution is contacted with the channel for about 8 hour to about 16 hours at from about 15° C. to about 30° C.

33. The method of claim 29, wherein the solution is pulled into the channel for the contacting.

34. The method of claim 29, wherein the solution is flushed out of the channel after contacting and the channel is dried.


SEQUENCE LISTING (2 sequences)

SEQ ID NO: 1 - DNA, 20 bp, Homo sapiens
caacttcatccacgttcacc

SEQ ID NO: 2 - DNA, 20 bp, Homo sapiens
acacaactgtgttcactagc